(19) United States
(12) Patent Application Publication
(10) Pub. No.: US 2025/0320010 A1
(43) Pub. Date: Oct. 16, 2025

(54) WRAPPING APPARATUS

(71) Applicant: ARMAN TAJMIRRIAHI, ISFAHAN (IR)

(72) Inventor: ARMAN TAJMIRRIAHI, ISFAHAN (IR)

(21) Appl. No.: 19/183,552

(22) Filed: Apr. 18, 2025

Related U.S. Application Data
(63) Continuation-in-part of application No. PCT/IB2022/059999, filed on Oct. 18, 2022.

(51) Int. Cl. B65B 31/04; B65B 41/02; B65B 55/16; B65B 61/06

(52) U.S. Cl. CPC B65B 31/048; B65B 41/02; B65B 55/16; B65B 61/06

(57) ABSTRACT

In accordance with some embodiments herein, a wrapping apparatus is provided. The wrapping apparatus includes a dispensing assembly and a vacuum sealing device. The dispensing assembly includes a sheet holder and a cutting device. The sheet holder is configured to hold a roll of sheet material and dispense a portion of the sheet material over a container. The cutting device is configured to separate a dispensed sheet including the portion of the sheet material from the roll of sheet material. The vacuum sealing device is configured to apply a suction force to a space between the dispensed sheet and the container to draw the dispensed sheet towards a surface of the container and/or an item in the container.

### RELATED APPLICATION

This application claims priority to and is a continuation-in-part of International Application Number PCT/IB2022/059999, filed on Oct. 18, 2022, entitled “A ROLL SHEET SAVING CONTAINER AND A ROLL SHEET SAVING MACHINE”, which is incorporated by reference herein in its entirety.

### TECHNICAL FIELD

The present disclosure relates to mechanical apparatuses, for example, to wrapping apparatuses.

### BACKGROUND

Wrapping apparatuses may be used in various applications, such as food industry, packaging, etc. A wrapping apparatus may be used for hygienically covering reusable dishware, such as plates, with a film, such as cellophane. After food consumption, the film may be removed and discarded, leaving the underlying plate clean and ready for reuse without washing. This system enables sanitary, time-saving meal serving and post-meal cleanup, suitable for households, restaurants, or institutional food services.

### SUMMARY

This summary is provided to introduce a selection of concepts in a simplified form that are further described below in the detailed description. This summary is not intended to identify key factors or essential features of the claimed subject matter, nor is it intended to be used to limit the scope of the claimed subject matter.

In some embodiments, a wrapping apparatus is provided. The wrapping apparatus includes a dispensing assembly and a vacuum sealing device. The dispensing assembly includes a sheet holder and a cutting device. The sheet holder is configured to hold a roll of sheet material and dispense a portion of the sheet material over a container. The cutting device is configured to separate a dispensed sheet including the portion of the sheet material from the roll of sheet material. The vacuum sealing device is configured to apply a suction force to a space between the dispensed sheet and the container to draw the dispensed sheet towards a surface of the container and/or an item in the container. In some embodiments, the wrapping apparatus includes an ultraviolet (UV) sterilizer device. The UV sterilizer device includes a UV container defining a UV chamber configured to store one or more utensils and one or more UV lamps configured to emit UV light to disinfect the one or more utensils.

### BRIEF DESCRIPTION OF DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1A illustrates a wrapping apparatus comprising a dispensing assembly in an open-lid state, a vacuum sealing device, a heat blower sealing device and a UV sterilizer device in a closed-lid state, in accordance with some embodiments.

FIG. 1B illustrates a wrapping apparatus comprising a dispensing assembly in a closed-lid state, a vacuum sealing device and a UV sterilizer device in a closed-lid state, in accordance with some embodiments.

FIG. 1C illustrates a dispensing assembly in an open-lid state and a UV sterilizer device in a closed-lid state, in accordance with some embodiments.

FIG. 1D illustrates an image of assembling different components of a sheet holder, in accordance with some embodiments.

FIG. 1E illustrates a back view of a dispensing assembly in an open-lid state, in accordance with some embodiments.

FIG. 1F illustrates a 2-dimensional front view of a dispensing assembly in a closed-lid state, in accordance with some embodiments.

FIG. 1G illustrates a 2-dimensional exploded view of a dispensing assembly and a UV sterilizer device, in accordance with some embodiments.

FIG. 1H illustrates an image showing how to pull a dispensed sheet out of a dispensing assembly by a user who is pulling out the dispensed sheet with both hands, in accordance with some embodiments.

FIG. 1I illustrates different components of a container holder, in accordance with some embodiments.

FIG. 2A illustrates a portable wrapping apparatus comprising a dispensing assembly in an open-lid state, a vacuum sealing device and a sheet pulling grip, in accordance with some embodiments.

FIG. 2B illustrates a 2-dimensional top view of a portable wrapping apparatus comprising a portable dispensing assembly in an open-lid state, a vacuum sealing device and a sheet pulling grip, in accordance with some embodiments.

FIG. 2C illustrates a sheet pulling grip in open-blade mode and in closed-blade mode, in accordance with some embodiments.

FIG. 2D illustrates a sheet pulling grip in open-clip mode and in closed-clip mode, in accordance with some embodiments.

FIG. 3 illustrates an automated wrapping apparatus comprising an automated dispensing assembly, an automated vacuum sealing device and a UV sterilizer device in an open-lid state, in accordance with some embodiments.

FIG. 4 illustrates an image of a toilet equipped with a wrapping apparatus comprising a dispensing assembly in an open-lid state and a vacuum sealing device, in accordance with some embodiments.

FIG. 5 illustrates a multifunctional wrapping apparatus comprising a plurality of dispensing assemblies for cutting and wrapping different sheets, in accordance with some embodiments.

### DETAILED DESCRIPTION

Subject matter will now be described more fully hereinafter with reference to the accompanying drawings, which form a part hereof, and which show, by way of illustration, specific example embodiments. This description is not intended as an extensive or detailed discussion of known concepts. Details that are known generally to those of ordinary skill in the relevant art may have been omitted, or may be handled in summary fashion.

The following subject matter may be embodied in a variety of different forms, such as methods, devices, components, and/or systems. Accordingly, this subject matter is not intended to be construed as limited to any example embodiments set forth herein. Rather, example embodiments are provided merely to be illustrative.

The present disclosure provides a wrapping apparatus. In some embodiments, the wrapping apparatus may comprise a dispensing assembly, a vacuum sealing device and/or a heat blower sealing device. In some examples, the dispensing assembly may comprise a sheet holder and a cutting device. In some examples, the sheet holder may be configured to hold a roll of sheet material and may dispense a portion of the sheet material over a container. In some examples, the cutting device may be configured to separate a dispensed sheet, wherein the dispensed sheet may comprise the portion of the sheet material from the roll of sheet material. In some examples, the vacuum sealing device may be configured to apply a suction force to a space between the dispensed sheet and the container to draw the dispensed sheet towards a surface of the container and/or an item in the container. In some examples, the heat blower sealing device may be configured to apply a compression force to the dispensed sheet to push the dispensed sheet towards a surface of the container and/or an item in the container. Alternatively and/or additionally, the wrapping apparatus may comprise an ultraviolet (UV) sterilizer device. In some examples, the UV sterilizer device may comprise a UV container defining a UV chamber configured to store one or more utensils and may comprise one or more UV lamps configured to emit UV light to disinfect the one or more utensils.

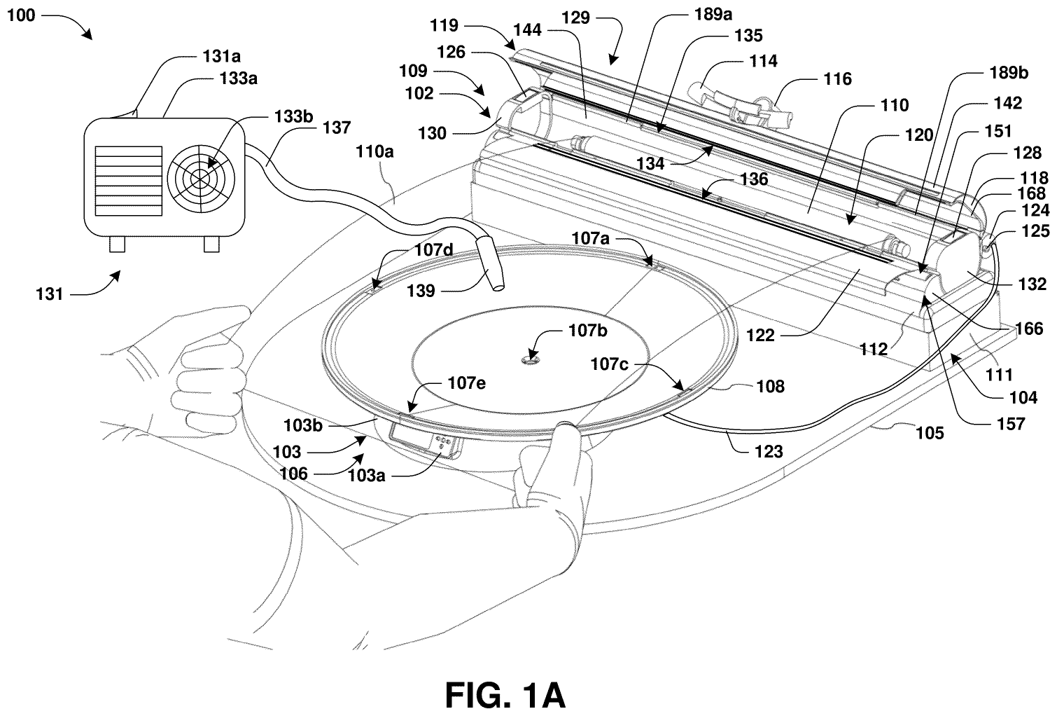

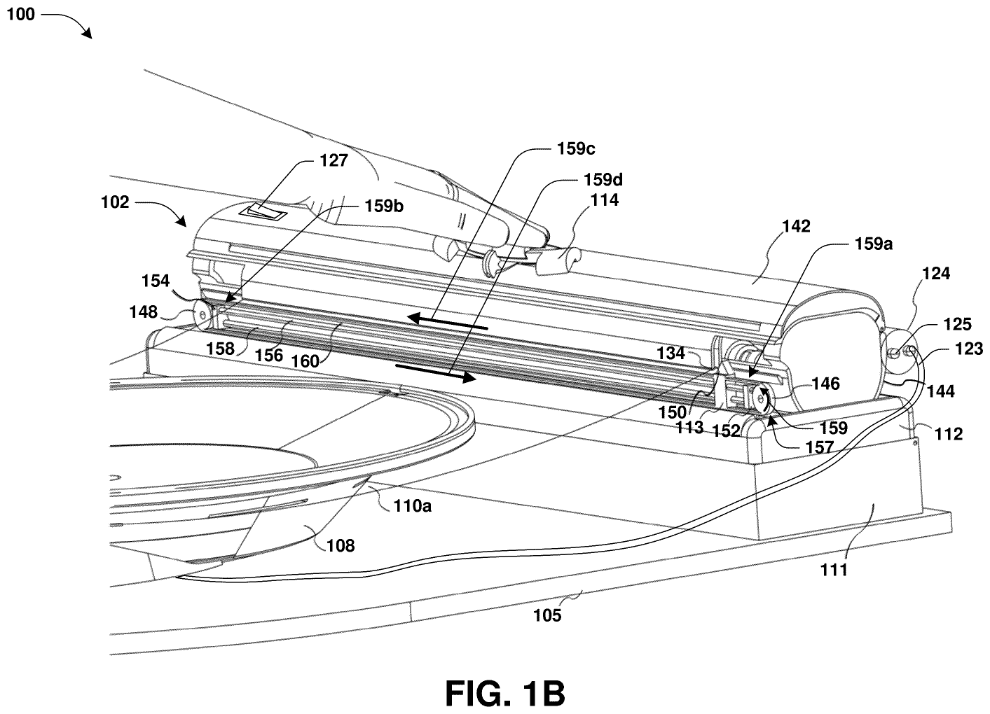

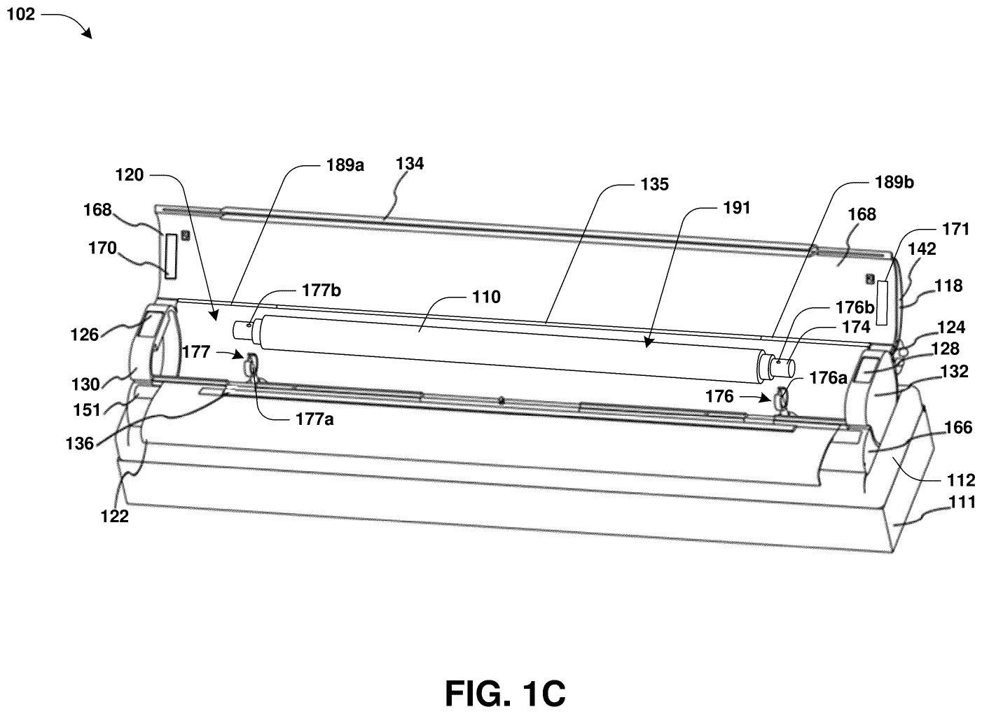

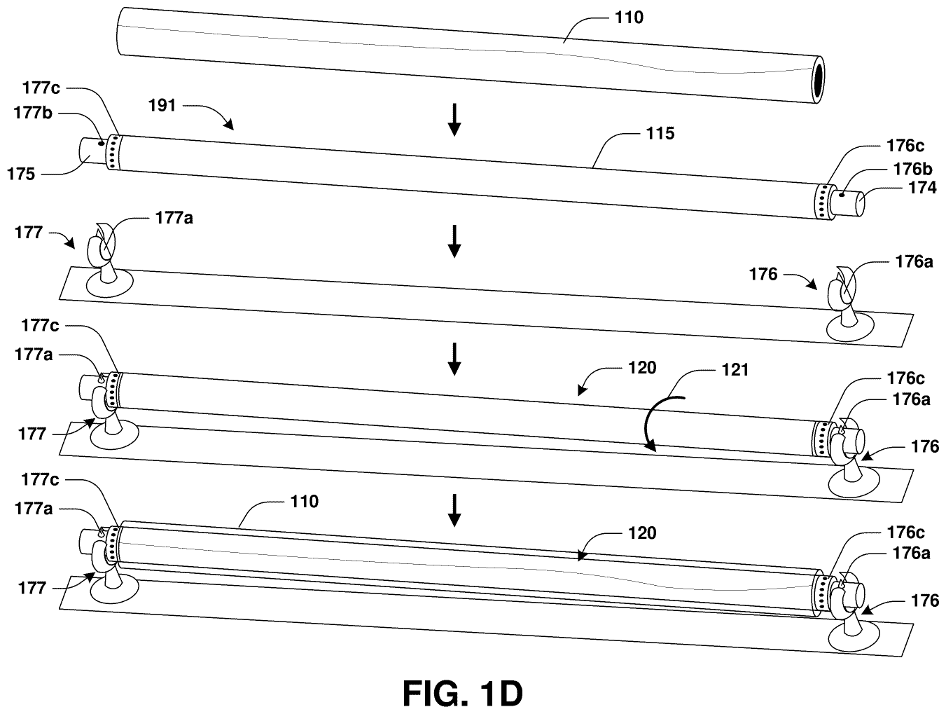

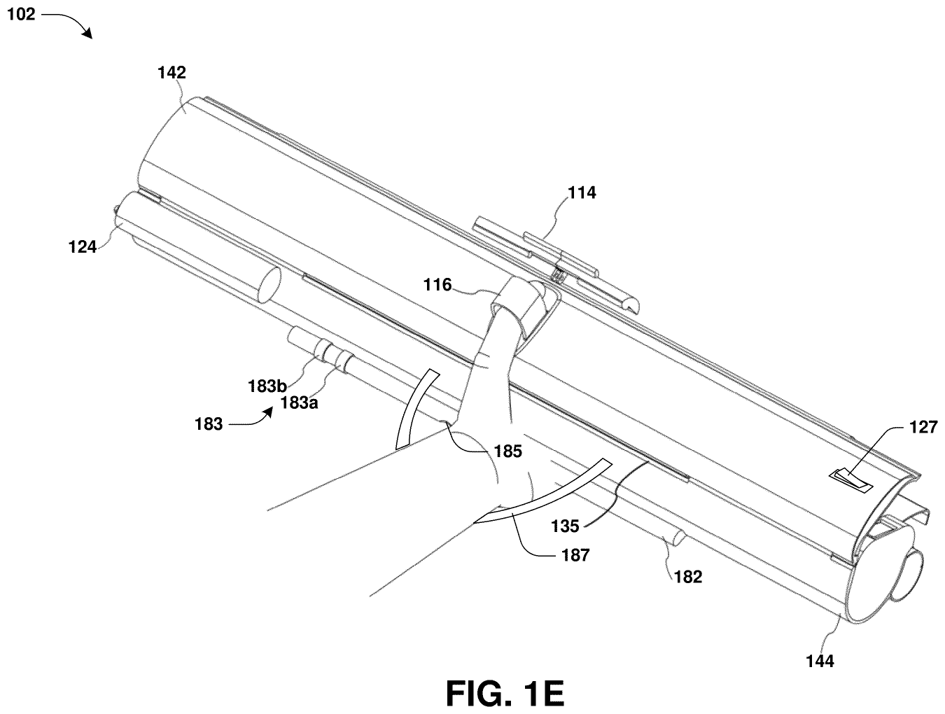

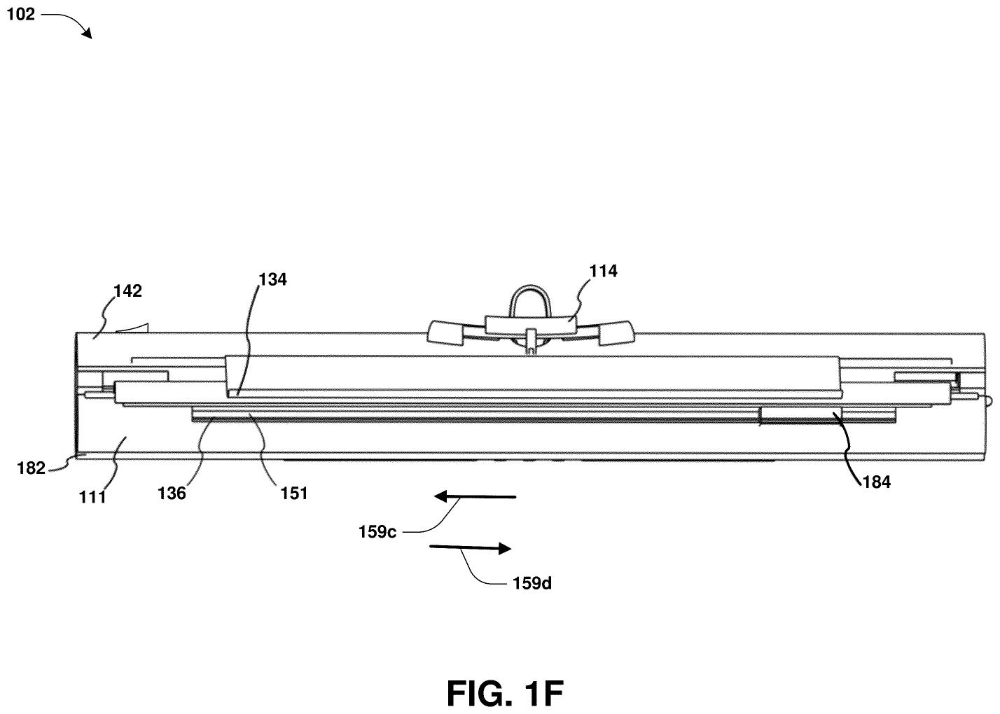

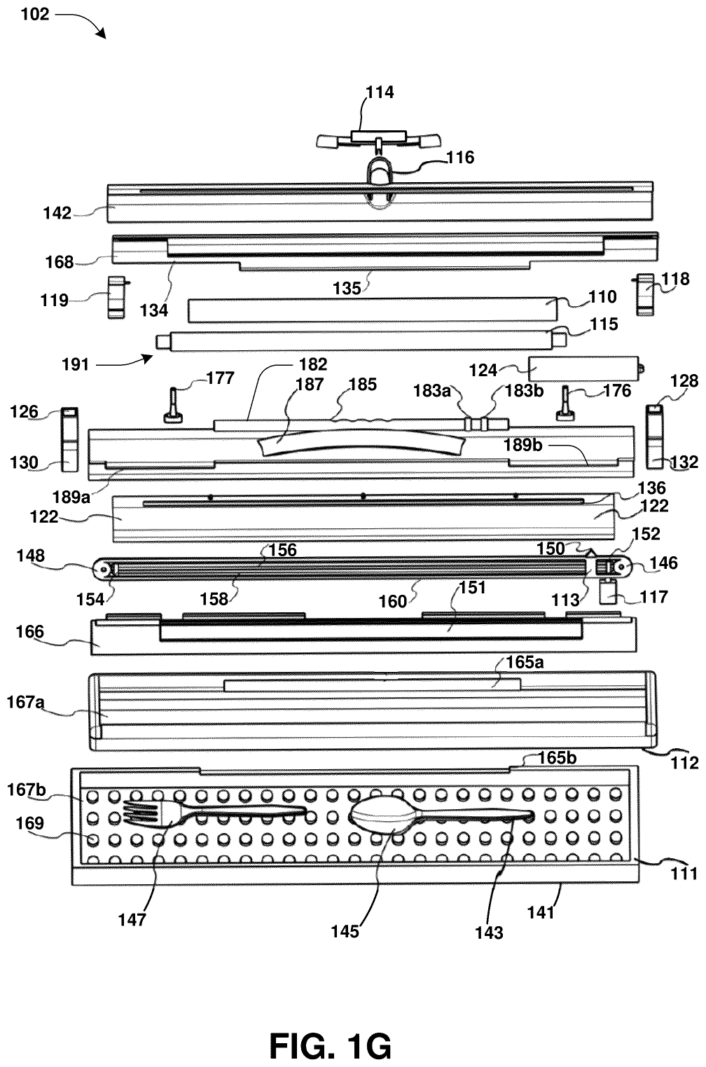

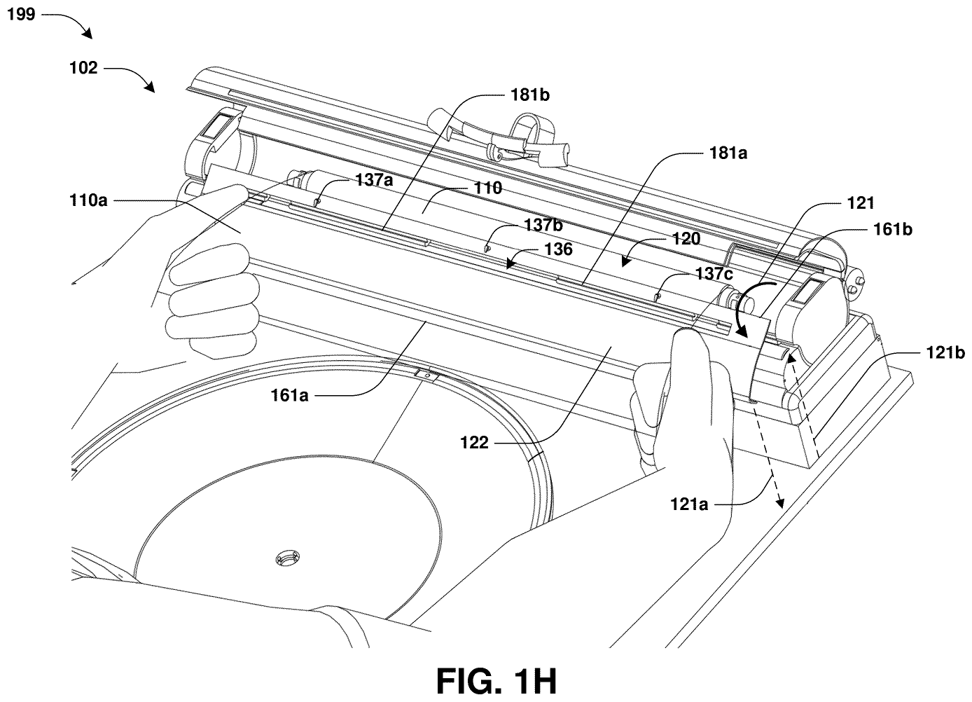

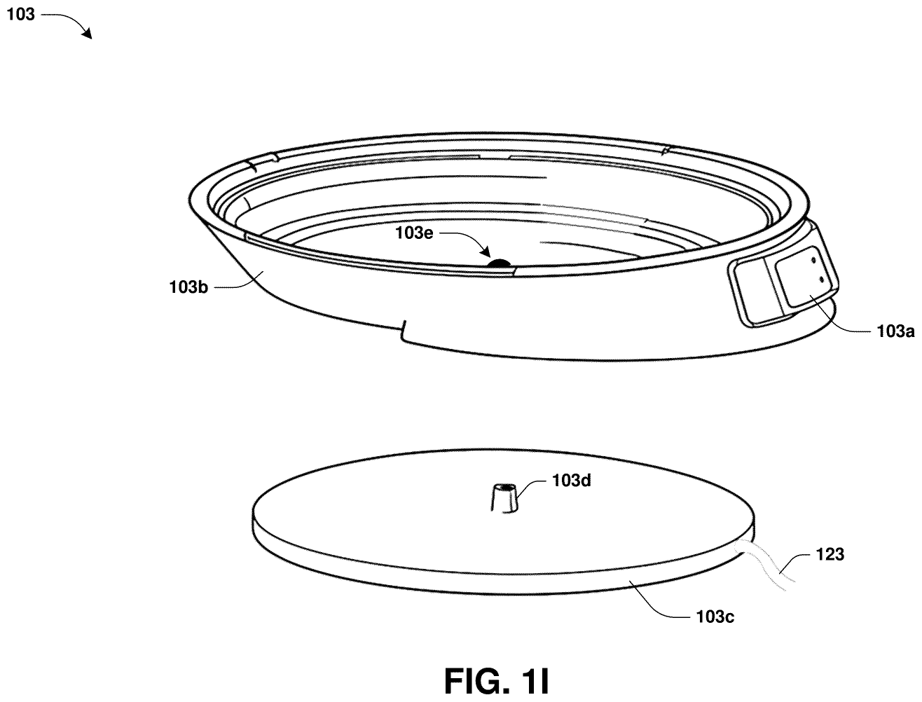

FIG. 1A illustrates a wrapping apparatus 100 comprising a dispensing assembly 102 in an open-lid state, a vacuum sealing device 106, a heat blower sealing device 131 and an ultraviolet (UV) sterilizer device 104 in a closed-lid state, in accordance with some embodiments. In some examples, the wrapping apparatus 100 may comprise the dispensing assembly 102, the vacuum sealing device 106, the heat blower sealing device 131, the UV sterilizer device 104 and/or a casing. In some examples, the dispensing assembly 102 may comprise a sheet holder 120 and/or a cutting device 157.

In some examples, the sheet holder 120 may be configured to hold a roll of sheet material 110 and dispense a portion of the sheet material over a container 108. In some examples, the sheet material may comprise plastic sheets, Aluminum foil sheets, paper-based sheets and/or biodegradable sheets. In some examples, the plastic sheets may comprise Polyethylene (PE) Sheets, Polypropylene (PP) Sheets, Polyethylene Terephthalate (PET) Sheets and/or Polyvinyl chloride (PVC) Sheets. In some examples, the paper-based sheets may comprise waxed paper sheets, greaseproof paper sheets and/or kraft paper sheets. In some examples, the biodegradable sheets may be compostable sheets and may comprise Polylactic Acid (PLA) Sheets and/or Cellulose/Cellophane Sheets. In some examples, the sheet material may comprise vacuum sealing sheets, silicone baking sheets and/or thermo-formable sheets. In some examples, the sheet material may comprise adhesive sheets and/or tapes. In some examples, the roll of sheet material 110 may comprise a central cavity (e.g., a central hole) and a roller may be inserted to the central cavity and/or may hold the roll of sheet material 110.

In some examples, the vacuum sealing device 106 may comprise a suction device (e.g., a pump 124), one or more suction tubes (e.g., a suction tube 123) and/or a holder (e.g., a container holder 103). In some examples, the vacuum sealing device 106 may be configured to apply a suction force to a space between a dispensed sheet 110a and the container 108 to draw the dispensed sheet 110a towards (e.g., against) at least one of a surface of the container 108 or an item (e.g., fruit, vegetables, food, etc.) in the container 108. In some examples, the vacuum sealing device 106 may be configured to remove air from between the dispensed sheet 110a and the surface of the container 108. In some examples, removing the air from between the dispensed sheet 110a and the surface of the container 108 may cause the dispensed sheet 110a to conform to the surface of the container 108 and/or the item in the container 108. In some examples, the suction tube 123 may connect the pump 124 to one or more holes (e.g., a first hole 107a, a second hole 107b, a third hole 107c, a fourth hole 107d, a fifth hole 107e) defined in the container 108. In some examples, the pump 124 may apply the suction force to the space between the dispensed sheet 110a and the container 108 via the suction tube 123. In some examples, the container holder 103 may comprise an input device 103a (e.g., a button, a switch, etc.) configured to trigger the pump 124 to apply the suction force in response to a user input. In some examples, the pump 124 may comprise an outlet 125 through which the air exits the pump 124.

In some examples, the heat blower sealing device 131 may comprise a blower (e.g., a compressor 133a), an air heater (e.g., a heating element 133b), an air nozzle (e.g., a hot air nozzle 139) and/or one or more compression tubes (e.g., a compression tube 137). In some examples, the heat blower sealing device 131 may be configured to apply a compression force to the dispensed sheet 110a to push the dispensed sheet 110a towards the surface of the container 108 and/or the item in the container 108. In some examples, the heat blower sealing device 131 may comprise an input device 131a (e.g., a button, a switch, etc.) configured to trigger the compressor 133a and/or the heating element 133b to apply the compression force via blowing a hot air in response to a user input. In some examples, the heating element 133b may apply heat to air inside the compressor 133a and the compressor 133a may blow the hot air towards the hot air nozzle 139 via the compression tube 137. In some examples, a user may position the hot air nozzle 139 above the container 108 to apply heat and/or to enable a secure attachment of the dispensed sheet 110a to the container 108.

In some examples, the wrapping apparatus 100 may comprise a tray 105. In some examples, at least a portion of the dispensing assembly 102, the vacuum sealing device 106, the heat blower sealing device 131, the UV sterilizer device 104 and/or the casing may be positioned on the tray 105. In some examples, the wrapping apparatus 100 may be fixed to the tray 105. In some examples, the wrapping apparatus 100 may be portable.

In some examples, the casing may be configured to house the dispensing assembly 102 and/or the cutting device 157. In some examples, the casing may comprise a lid 129 (e.g., a door, a cap, etc.), a chamber 166 (e.g., a housing), a lower covering 109 and/or a sheet guider plate 122. In some examples, the lid 129 may be configured to house a power supply (e.g., a battery), an electronic board (e.g., an electronic element) and/or a controller (e.g., a module, a control module, etc.) for operation of the wrapping apparatus 100. In some examples, the lid 129 may comprise a first layer 142 (e.g., a first plastic covering, a first plastic layer, etc.), a second layer 168 (e.g., a second plastic covering, a second plastic layer, etc.), a first side wall 118 (e.g., a first plastic side wall, etc.), a second side wall 119 (e.g., a second plastic side wall, etc.), a thumb grip 116 (e.g., a finger grip, a grip, etc.) and/or a handle 114 (e.g., a lever, etc.). In some examples, the lid 129 comprises a groove 134 (e.g., a slot, a trench, etc.) which may be configured to be in contact with the dispensed sheet 110a and may provide a path for a cutter to move and separate the dispensed sheet 110a. In some examples, the second layer 168 may comprise the groove 134 which may be configured to be in contact with the dispensed sheet 110a and may provide the path for the cutter to move and separate the dispense sheet 110a. In some examples, the lid 129 may be connected (e.g., attached) to the lower covering 109 via one or more hinges (e.g., a spring-loaded hinge 135) to provide a restoring force. In some examples, the first side wall 118 may connect at least a first portion of the first layer 142 to at least a first portion of the second layer 168. In some examples, the second side wall 119 may connect at least a second portion of the first layer 142 to at least a second portion of the second layer 168. In some examples, the thumb grip 116 may be placed on the first layer 142. In some examples, the handle 114 may be placed on the first layer 142. In some examples, the spring-loaded hinge 135 may comprise one or more hinge knuckles (e.g., a first hinge knuckle 189a, a second hinge knuckle 189b, etc.).

In some examples, the chamber 166 may be configured to house the cutting device 157. In some examples, the chamber 166 may comprise a first trench 151 allowing the cutter to move. In some examples, the sheet guider plate 122 may be configured to hold at least a portion of the dispensed sheet 110a of the roll of sheet material 110. In some examples, the sheet guider plate 122 may comprise a second trench 136 which may overly the first trench 151 of the chamber 166. In some examples, after closing the lid 129, the groove 134 may enter the second trench 136 and/or the second trench 136 may coincide and/or align with the first trench 151.

In some examples, the lower covering 109 may be configured to house the dispensing assembly 102 and/or the chamber 166. In some examples, the lower covering 109 may comprise a layer (e.g., a third layer 144, a third plastic covering and/or a third plastic layer, etc.) and one or more side walls (e.g., a third side wall 132, a third plastic side wall, a fourth side wall 130 and/or a third plastic side wall, etc.). In some examples, the third layer 144 may be connected (e.g., attached) to the lid 129 via the one or more hinges (e.g., the spring-loaded hinge 135). In some examples, the third side wall 132 may be connected (e.g., attached) to a first portion of the third layer 144. In some examples, the fourth side wall 130 may be connected (e.g., attached) to a second portion of the third layer 144. In some examples, the casing may comprise one or more locks (e.g., one or more male-female locks, one or more fasteners, one or more magnetic locks, a first male-female lock, a second male-female lock, etc.) configured to attach and/or lock the lid 129 to the lower covering 109. In some examples, the third side wall 132 may comprise a first receiving element 128 (e.g., a socket, a slot, etc.). In some examples, the fourth side wall 130 may comprise a second receiving element 126. In some examples, the lid 129 may comprise a first protruding element (e.g., a plug, a pin, etc.) and/or a second protruding element. For examples, after closing the lid 129, the first protruding element may enter the first receiving element 128 and the second protruding element may enter the second receiving element 126. In some examples, the first receiving element 128 may lock (e.g., hold, keep, etc.) the first protruding element and/or the second receiving element 126 may lock (e.g., hold, keep, etc.) the second protruding element. In some examples, the handle 114 may be connected to the first protruding element, the second protruding element, the first receiving element 128 and/or the second receiving element 126. For example, after pressing (e.g., triggering) the handle 114, the first receiving element 128 may disengage (e.g., release) the first protruding element and/or the second receiving element 126 may disengage (e.g., release) the second protruding element. After disengaging the first protruding element and the second protruding element, the spring-loaded hinge 135 may apply the restoring force to separate the lid 129 from the lower covering 109 and/or to open the lid 129.

In some examples, the UV sterilizer device 104 may comprise a UV container and/or one or more UV lamps. In some examples, the UV container may comprise a UV container body 111 and/or a UV container lid 112. In some examples the UV container body 111 may define a UV chamber configured to store one or more utensils (e.g., one or more kitchenware, one or more spoons, one or more forks, one or more knives, etc.). In some examples, the one or more UV lamps may be configured to emit UV light to disinfect (e.g., sterilize) the one or more utensils. In some examples, at least one UV lamp of the one or more UV lamps may be placed (e.g., mounted) inside the UV container body 111. In some examples, at least one UV lamp of the one or more UV lamps may be placed (e.g., mounted) inside the UV container lid 112. In some examples, a wavelength range of the UV light may be between about 100 nanometers to about 400 nanometers. In some examples, a wavelength range of the UV light may be between about 200 nanometers to about 209 nanometers.

FIG. 1B illustrates the wrapping apparatus 100 comprising the dispensing assembly 102 in a closed-lid state, the vacuum sealing device 106 and the UV sterilizer device 104 in a closed-lid state, in accordance with some embodiments. In FIG. 1B, the sheet guider plate 122 and the chamber 166 are not shown on the wrapping apparatus 100 in order to make components of the cutting device 157 visible. In some examples, the cutting device 157 may comprise the cutter (shown with reference number 150), a cutter driving mechanism and/or an input device 127 (e.g., a button, a switch, etc.). In some examples, the input device 127 may be configured to trigger (e.g., run, initiate, start, command, etc.) the cutter driving mechanism to move the cutter 150 across an extent of the sheet material to separate the dispensed sheet 110a from the roll of sheet material 110 in response to a user input. In some examples, the cutter 150 may be a double-edge blade configured to separate the dispensed sheet 110a in a reciprocating path (e.g., a bilateral direction, a first direction 159c and a second direction 159d, etc.). In some examples, the cutter 150 may be a circular cutter, a straight cutter (e.g., single edge cutter), and/or a serrated cutter. In some examples, the cutter 150 may be made of Carbon Steel, Stainless Steel, High-Speed Steel (HSS), Tool Steel, Ceramic, Tungsten Carbide, Diamond-Coated, Plastic and Composite Materials Aluminum Alloys and/or Copper Alloys.

In some examples, the cutter 150 may start to move from a first point 159a to a second point 159b in the first direction 159c and may move back from the second point 159b to the first point 159a in the second direction 159d (e.g., a direction opposite of the first direction 159c).

In some examples, the cutter driving mechanism may comprise one or more pulleys (e.g., a first pulley 146, a second pulley 148, etc.), a conveyor belt 160 (e.g., a chain, a strap, etc.), a wagon 113 (e.g., a carrier, etc.), a rail (e.g., a pathway), one or more sensors (e.g., one or more proximity sensors, a first sensor 152, a second sensor 154, etc.) and/or a motor (e.g., an electric motor, etc.). In some examples, the conveyor belt 160 may be configured to connect the first pulley 146 to the second pulley 148. In some examples, the wagon 113 may be coupled to the cutter 150 via one or more bearings. In some examples, the rail may comprise a first guide rod 156 and a second guide rod 158, wherein the rail may provide the reciprocating path for the wagon 113 and the cutter 150. In some examples, the first sensor 152 may be connected to a first side of the rail and/or the second sensor 154 may be connected to a second side of the rail. In some examples, the electric motor may be configured to rotate the first pulley 146 and/or to rotate the conveyor belt 160 in a first rotational direction 159 and a second rotational direction (e.g., a rotational direction opposite to the first rotational direction 159). In some examples, the rotation of the conveyor belt 160 may move the wagon 113 and/or the cutter 150 in the reciprocating path. In some examples, the rotation of the first pulley 146 around the first rotational direction 159 may cause the conveyor belt 160 to rotate around the first rotational direction 159 and the rotation of the conveyor belt 160 around the first rotational direction 159 may cause the second pulley 148 to rotate around the first rotational direction 159. In some examples, rotation of the first pulley 146 around the second rotational direction may cause the conveyor belt 160 to rotate around the second rotational direction and the rotation of the conveyor belt 160 around the second rotational direction may cause the second pulley 148 to rotate around the second rotational direction. In some examples, the wagon 113 may comprise one or more bearings (e.g., one or more linear bearings) connected to the rail. The wagon 113 may be fixed (e.g., attached) to the cutter 150 and/or to the conveyor belt 160. Upon rotation of the conveyor belt 160 around the first rotational direction 159, the wagon 113 and/or the cutter 150 may move in the first direction 159c. Upon rotation of the conveyor belt 160 around the second rotational direction, the wagon 113 and/or the cutter 150 may move in the second direction 159d. In some examples, the first sensor 152 and the second sensor 154 may be connected to the electronic board and may transmit one or more signals (e.g., one or more proximity signals) to the electronic board. For example, upon arrival of (e.g., approaching) the wagon 113 to the second sensor 154, the second sensor 154 may sense the wagon 113 and transmit a first signal (e.g., a first proximity signal) to the electronic board. After transmitting the first signal, the electronic board may transmit a second signal to the controller, and the controller may cause the electric motor to rotate the first pulley 146 around the second rotational direction. The rotation of the first pulley 146 around the second rotational direction may cause the wagon 113 and/or the cutter 150 to move from the second point 159b to the first point 159a (e.g., a point close to the first sensor 152) in the second direction 159d. Upon arrival of (e.g., approaching) the wagon 113 and/or the cutter 150 to the first sensor 152, the first sensor 152 may sense the wagon 113 and transmit a third signal (e.g., a third proximity signal) to the electronic board. After transmitting the third signal, the electronic board may transmit a fourth signal to the controller, and the controller may cause the electric motor to stop working (e.g., turn off). In some examples, after transmitting the fourth signal, the electronic board may turn on a lamp (e.g., a light emitting diode (LED)) and/or a beeper (e.g., a buzzer) for a period of time to alarm a user. In some examples, the lamp and/or the beeper may indicate turning off the electric motor and/or finishing the cutting operation.

FIG. 1C illustrates the dispensing assembly 102 in an open-lid state and the UV sterilizer device 104 in a closed-lid state, in accordance with some embodiments. As shown in FIG. 1C, the lid 129 is open wherein the first protruding element (shown with reference number 171) is disengaged from the first receiving element (shown with reference number 128) and the second protruding element (shown with reference number 170) is disengaged from the second receiving element (shown with reference number 126). In some examples, the sheet holder 120 may comprise a roller 191, a first clip 176 and/or a second clip 177. In some examples, at least a portion of the roller 191 may be inserted into the central cavity of the roll of sheet material 110 and/or may hold the roll of sheet material 110. In some examples, the first clip 176 and/or the second clip 177 may hold the roller 191. In some examples, the roller 191 may be configured to rotate the roll of sheet material 110 and/or to open a portion of the sheet material. In some examples, the first clip 176 may be configured to hold at least a first portion of the roller 191. In some examples, the second clip 177 may be configured to hold at least a second portion of the roller 191.

FIG. 1D illustrates an image of assembling different components of the sheet holder 120, in accordance with some embodiments. In some examples, the roller 191 may comprise one or more fixed rods (e.g., a first fixed rod 174, a second fixed rod 175) and/or one or more rotatable rods (e.g., a rotatable rod 115). In some examples, the first fixed rod 174 may comprise one or more holes (e.g., a first hole 176b). In some examples, the first fixed rod 174 may be coupled (e.g., connected) to one or more bearings (e.g., one or more ball bearings, one or more roll bearings, a first bearing 176c, etc.). In some examples, the second fixed rod 175 may comprise one or more holes (e.g., a second hole 177b). In some examples, the second fixed rod 175 may be coupled (e.g., connected) to one or more bearings (e.g., one or more ball bearings, one or more roll bearings, a second bearing 177c, etc.). In some examples, the rotatable rod 115 may be coupled to the first bearing 176c from one side and/or may be coupled to the second bearing 177c from the other side. In some examples, the rotatable rod 115 may be configured to hold the roll of sheet material 110. In some examples, the first clip 176 may comprise one or more spikes (e.g., a first spike 176a, etc.). In some examples, the first spike 176a may enter the first hole 176b and/or may hold the first fixed rod 174. In some examples, the second clip 177 may comprise one or more spikes (e.g., a second spike 177a, etc.). In some examples, the second spike 177a may enter the second hole 177b and/or may hold the second fixed rod 175. In some examples, the first spike 176a may be provided to prevent rotation of the first fixed rod 174 and the second spike 177a may be provided to prevent rotation of the second fixed rod 175. In some examples, the first bearing 176c and/or the second bearing 177c may cause the rotatable rod 115 to rotate around a third rotational direction 121. In some examples, the first bearing 176c and/or the second bearing 177c may cause the rotatable rod 115 to rotate around a fourth rotational direction (e.g., a rotational direction opposite to the third rotational direction 121). In some examples, a user may hold a portion of the sheet material and/or may pull out the portion of the sheet material. After pulling the portion of the sheet material out, the roller 191 may rotate around the third rotational direction 121 or the fourth rotational direction. Alternatively and/or additionally, the rotation of the roller 191 may cause the roll of sheet material 110 to rotate around the third rotational direction 121 or the fourth rotational direction. Alternatively and/or additionally, the rotation of the roll of sheet material 110 may cause an extent of the sheet material to be dispensed.

FIG. 1E illustrates a back view of the dispensing assembly 102 in an open-lid state, in accordance with some embodiments. In some examples, the dispensing assembly 102 may comprise a bar 182 (e.g., a knurl, a knurled bar, a grip bar, a friction bar, etc.) wherein the bar 182 may comprise a series of small ridges 185 (e.g., diamonds) that may be pressed (e.g., cut) into the bar 182 to improve grip and friction. In some examples, the bar 182 may comprise charging contacts 183 (e.g., battery terminals, a first charging contact 183a, a second charging contact 183b, etc.). In some examples, after discharging the power supply (e.g., the battery), the charging contacts 183 may be connected to an external power source (e.g., an external battery, etc.) to initiate (e.g., start) recharging the power supply. In some examples, the power supply may be required to operate (e.g., run, drive, energize, etc.) the dispensing assembly 102, the vacuum sealing device 106, the electronic board, the controller and/or the heat blower sealing device 131. In some examples, a user's thumb may be inserted into the thumb grip 116 and the user's palm may be in contact with at least a portion (e.g., a ridged portion, a diamond portion, etc.) of the bar 182. In some examples, the bar 182 may serve as an interface between the casing and the UV container. In some examples, the bar 182 may serve as an interface between the lower covering 109 and the UV container lid 112. In some examples, the bar 182 may function as a part of a hinge mechanism between the lower covering 109, the UV container lid 112 and/or the UV container body 111. In some examples, the lower covering 109 may comprise a safety strap 187 (e.g., a wrist strap) which may be configured to prevent the dispensing assembly 102 from falling out of the user's hand.

FIG. 1F illustrates a 2-dimensional front view of the dispensing assembly 102 in a closed-lid state, in accordance with some embodiments. In some examples, a handle 184 may be connected (e.g., attached) to the cutter 150 for manual operation of the cutter 150. In some examples, a user may hold the handle 184 and may move the handle 184 in the first direction 159c and/or in the second direction 159d. Alternatively and/or additionally, the moving the handle 184 may allow the cutter 150 to move in the first direction 159c and/or the second direction 159d.

FIG. 1G illustrates a 2-dimensional exploded view of the dispensing assembly 102 and the UV sterilizer device 104, in accordance with some embodiments. As shown in FIG. 1G, the electric motor (shown with reference number 117) is connected to the first pulley 146. In some examples, the electric motor may be connected to the first pulley 146 via one or more gears which may be configured to rotate the first pulley 146. In some examples, the UV container lid 112 may comprise an upper casing 167a, a lower casing (not shown) and/or a first hinge portion 165a. In some examples, the UV container body 111 may comprise a UV housing 141 and/or a second hinge portion 165b. In some examples, the UV housing 141 may comprise a first base 167b (e.g., a lamp platform, etc.) which may be configured to hold (e.g., support) a first plurality of UV lamps (e.g., a first UV lamp 169, etc.). In some examples, the UV container lid 112 may comprise a second base (not shown) which may be configured to hold a second plurality of UV lamps. In some examples, the first base 167b and/or the second base may be configured to store one or more utensils (e.g., a spoon 145, a fork 147, etc.). In some examples, the first base 167b and/or the second base may comprise one or more clips (e.g., one or more utensils clips, a first utensil clip 143) configured to hold the one or more clips inside the UV sterilizer device 104. In some examples, the first hinge portion 165a may be connected (e.g., attached) to the second hinge portion 165b via the hinge mechanism.

FIG. 1H illustrates an image 199 showing how to pull the dispensed sheet 110a out of the dispensing assembly 102 by a user who is pulling out the dispensed sheet 110a with both hands, in accordance with some embodiments. In some examples, the sheet guider plate 122 may comprise one or more prongs (e.g., a first prong 137a, a second prong 137b, a third prong 137c, etc.) configured to hold (e.g., grip) a portion of the dispensed sheet 110a. In some examples, the sheet guider plate 122 may comprise one or more hinges (e.g., one or more spring-loaded hinges, a first hinge 181a, a second hinge 181b, etc.). In some examples, a user may hold a first portion of the dispensed sheet 110a by left hand and/or a second portion of the dispensed sheet 110a by right hand. While holding the dispensed sheet 110a, the user may push the sheet guider plate 122 towards a third direction 121a. Alternatively and/or additionally, the pushing the sheet guider plate 122 may cause the sheet guider plate 122 to rotate around the third rotational direction 121. Alternatively and/or additionally, the rotation may cause a first edge 161a of the sheet guider plate 122 to move towards the third direction 121a and may cause a second edge 161b of the sheet guider plate 122 to move towards a fourth direction 121b (e.g., a direction opposite to the third direction 121a). Alternatively and/or additionally, the movement of the second edge 161b, may detach the dispensed sheet 110a from the one or more prongs and the user may be able to pull the dispensed sheet 110a out of the dispensing assembly 102. In some examples, after releasing the sheet guider plate 122, the one or more hinges may cause the sheet guider plate 122 to rotate around the fourth rotational direction (e.g., opposite to the third rotational direction 121) and/or may return (e.g., bring back, restore, etc.) the sheet guider plate 122 to its initial state.

FIG. 1I illustrates different components of the container holder 103, in accordance with some embodiments. In some examples, the container holder 103 may comprise a base plate 103c configured to be connected to the suction tube 123. In some examples, the base plate 103c may comprise a nipple 103d (e.g., a short pipe, etc.). In some examples, the container holder 103 may comprise a pan 103b (e.g., a drain pan, a tray, etc.). In some examples, the container holder 103 may comprise a hole 103e (e.g., an outlet, an output, etc.). In some examples, the pan 103b may be placed over the base plate 103c. In some examples, after placing the pan 103b over the base plate 103c, the nipple 103d may enter the hole 103e and/or may fit onto the hole 103e. In some examples, the container holder 103 may comprise the input device 103a (e.g., the button, the switch, etc.) configured to trigger the pump 124 to apply the suction force in response to a user input. In some examples, a container may be placed over the pan 103b and/or may fit onto the pan 103b. In some examples, a user may trigger the input device 103a (e.g., the button, the switch, etc.). Alternatively and/or additionally, the triggering may allow the pump 124 to turn on (e.g., start). The pump 124 may apply the suction force to the space between the dispensed sheet 110a and a container via the suction tube 123. In some examples, applying the suction force, air between the dispensed sheet 110a and a top surface of the container may exit through a plurality of holes defined in the container and/or may enter the pan 103b. Alternatively and/or additionally, the air may exit the pan 103b via the hole 103e and/or may enter the base plate 103c via the nipple 103d. At least a portion of the air, after entering the base plate 103c, may enter the pump 124 via the suction tube 123 and/or exit the pump 124 via the outlet 125.

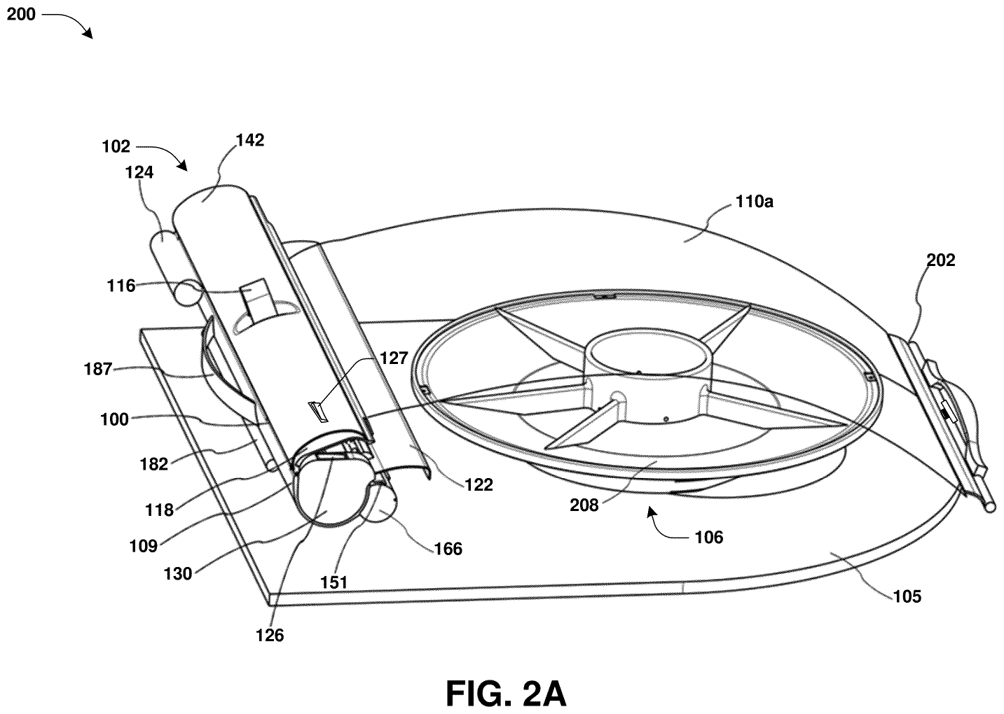

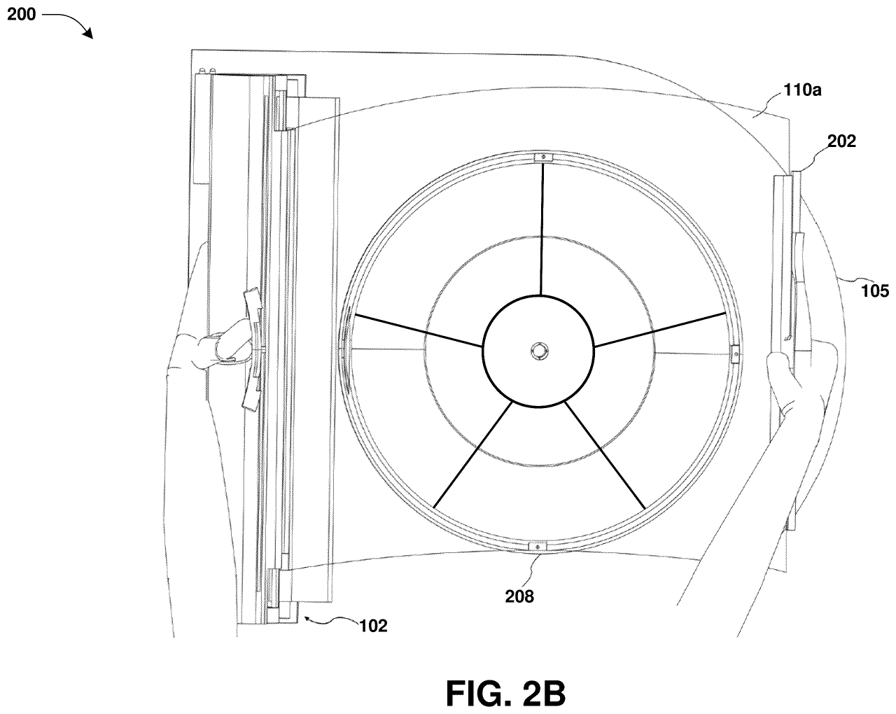

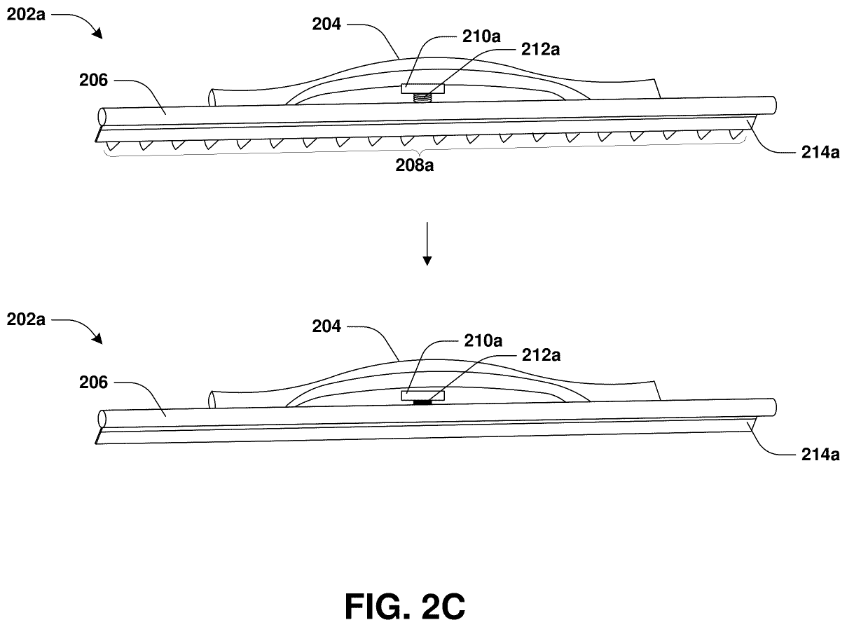

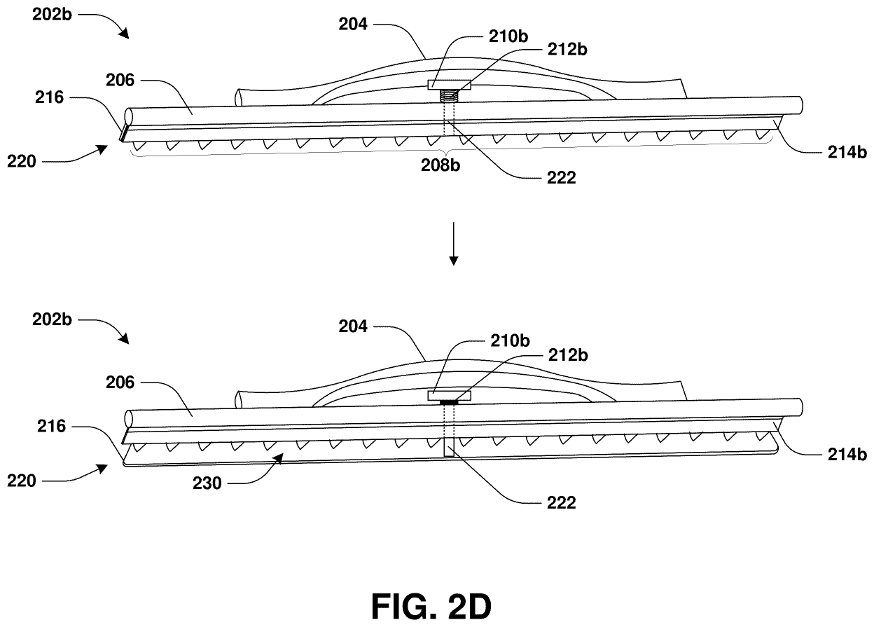

FIG. 2A illustrates a portable wrapping apparatus 200 comprising the dispensing assembly 102 in an open-lid state, the vacuum sealing device 106 and a sheet pulling grip 202, in accordance with some embodiments. FIG. 2B illustrates a 2-dimensional top view of the portable wrapping apparatus 200 comprising the dispensing assembly 102 in an open-lid state, the vacuum sealing device 106 and the sheet pulling grip 202, in accordance with some embodiments. FIG. 2C illustrates a first sheet pulling grip 202a in open-blade mode and in closed-blade mode, in accordance with some embodiments. FIG. 2D illustrates a second sheet pulling grip 202b comprising a clip 220 in open mode and closed mode, in accordance with some embodiments.

In some examples, the first sheet pulling grip 202a may comprise a hand grip 204, a sheet straightener roller 206, a first plurality of blades 208a and/or a first flat surface 214a (e.g., a body of the first sheet pulling grip 202a). In some examples, the first flat surface 214a may comprise a trench to house the first plurality of blades 208a. In some examples, the first sheet pulling grip 202a may be configured to pull the dispensed sheet 110a. In some examples, the sheet straightener roller 206 may be coupled to the hand grip 204 and/or the first flat surface 214a. In some examples, the sheet straightener roller 206 may be configured to flatten the dispensed sheet 110a. In some examples, a user may rub the sheet straightener roller 206 on the dispensed sheet 110a and may flatten the dispensed sheet 110a. In some examples, the first sheet pulling grip 202a may comprise a first button 210a, a first spring 212a and/or a first rod (not shown) connecting the first spring 212a and/or the first button 210a to the first plurality of blades 208a. In some examples, the user may hold the first sheet pulling grip 202a and may hold (e.g., grip) a portion (e.g., an edge) of the dispensed sheet 110a to pull the dispensed sheet 110a out of the sheet holder 120. In some examples, the first plurality of blades 208 may hold (e.g., grip) the portion of the dispensed sheet 110a. In some examples, the user may push the first button 210a and/or the first spring 212a. After pushing the first button 210a and/or the first spring 212, the first rod may pull the first plurality of blades 208 inside the trench. Alternatively and/or additionally, the pulling the first plurality of blades 205 inside the trench may cause the first plurality of blades 205 to detach the dispensed sheet 110a.

As shown in FIG. 2D, the second sheet pulling grip 202b may comprise the clip 220 configured to hold (e.g., grip) the portion of the dispensed sheet 110a. In some examples, the clip 220 may comprise a bladed surface 214b (e.g., a serrated surface) an/or a second flat surface 216. In some examples, the bladed surface 214b may comprise a second plurality of blades 208b. In some examples, a first side of the bladed surface 214b may be connected to a first side of the second flat surface 216 using a spring-loaded hinge (not shown). In some examples, the spring-loaded hinge may attach a second side of the bladed surface 214b to a second side of the second flat surface 216. In some examples, the bladed surface 214b may be disposed opposite the second flat surface 216. In some examples, the second sheet pulling grip 202b may comprise a second button 210b, a second spring 212b and/or a second rod 222. In some examples, the second button 210b, the second spring 212b and/or the second rod 222 may be connected to the spring-loaded hinge and/or may be connected to the second flat surface 216. In some examples, pushing the second button 210b may allow the second rod 222 to push the second flat surface 216 and/or to apply pressure to the spring-loaded hinge. In some examples, pushing the second button 210b may allow the bladed surface 214b to detach the second flat surface 216. A user may put a portion of the dispensed sheet 110a in a space 230 between the bladed surface 214b and the second flat surface 216. After putting the portion of the dispensed sheet 110a in the space 230, the user may release the second button 210b. After releasing the second button 210, the second side of the bladed surface 214b may attach to the second side of the second flat surface 216 and/or may hold (e.g., grip) the portion of the dispensed sheet 110a.

In some examples, a user may use the first sheet pulling grip 202a and/or the second sheet pulling grip 202b to hold a portion of the dispensed sheet 110a. While holding the dispensed sheet 110a, the user may push the sheet guider plate 122 towards the third direction 121a. Alternatively and/or additionally, the pushing the sheet guider plate 122 may cause the sheet guider plate 122 to rotate around the third rotational direction 121. Alternatively and/or additionally, the rotation may cause a first edge 161a of the sheet guider plate 122 to move towards the third direction 121a and may cause a second edge 161b of the sheet guider plate 122 to move towards the fourth direction 121b (e.g., a direction opposite to the third direction 121a). Alternatively and/or additionally, the movement of the second edge 161b, may detach the dispensed sheet 110a from the one or more prongs and the user may be able to pull the dispensed sheet 110a out of the dispensing assembly 102.

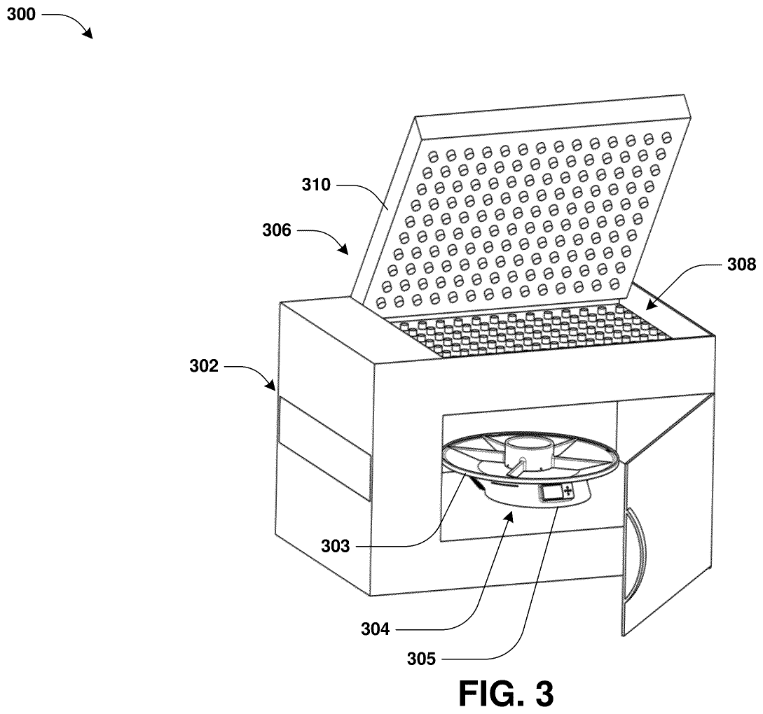

FIG. 3 illustrates an automated wrapping apparatus 300 comprising an automated dispensing assembly 302, an automated vacuum sealing device 304 and a UV sterilizer device 306 in an open-lid state, in accordance with some embodiments. In some examples, the automated dispensing assembly 302 may be placed within one or more walls of the automated wrapping apparatus 300. In some examples, the automated dispensing assembly 302 may comprise one or more robotic arms. In some examples, the one or more robotic arms may be used to wrap a dispensed sheet of a roll of sheet material) about a container 303 placed on a container holder 305. In some examples, the container holder 305 may be connected to a suction device (e.g., a pump) in which the suction device may use a suction force to wrap the container 305 by the dispensed sheet in response to a user input.

In some examples, the UV sterilizer device 306 may comprise a UV chamber 308 (e.g., a box), a movable lid 310 and/or one or more UV lamps. For example, the UV sterilizer device 306 may comprise one or more reflective materials (e.g., glass, aluminum foil, etc.). In some examples a UV container body may define the UV chamber 308 configured to store one or more utensils (e.g., one or more kitchenware, one or more spoons, one or more forks, one or more knives, etc.). In some examples, the one or more UV lamps may be configured to emit UV light to disinfect (e.g., sterilize) the one or more utensils. In some examples, at least one UV lamp of the one or more UV lamps may be placed (e.g., mounted) inside the UV chamber 308. In some examples, at least one UV lamp of the one or more UV lamps may be placed (e.g., mounted) inside the movable lid 310. In some examples, a wavelength range of the UV light may be between about 100 nanometers to about 400 nanometers. In some examples, a wavelength range of the UV light may be between about 200 nanometers to about 209 nanometers.

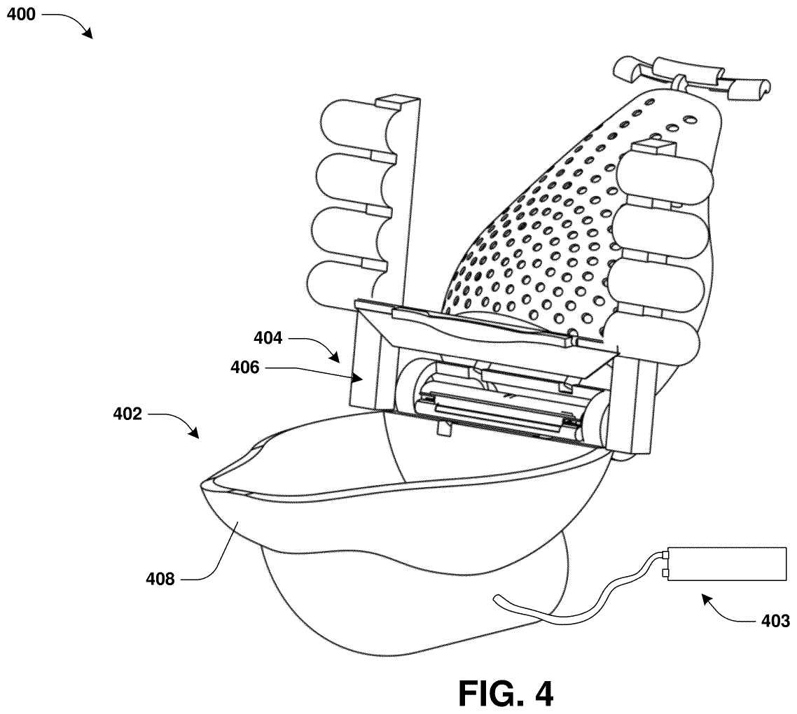

FIG. 4 illustrates an image 400 of a toilet 402 equipped with a wrapping apparatus 404 comprising a dispensing assembly 406 in an open-lid state and a vacuum sealing device 403, in accordance with some embodiments. In some examples, the dispensing assembly 406 may distribute a sheet material manually and/or automatically over a portion (e.g., inner surface) of a seat 408 associated with the toilet 402. In some examples, a user may pull the sheet material over the seat 408 manually and/or via using a sheet pulling grip. In some examples, the vacuum sealing device 403 may be used to vacuum air between the sheet material and the seat 408. In some examples, after the user used the toilet 402, the wrapping apparatus 404 may utilize one or more arms (e.g., robotic arms) to collect the sheet material and dispose the sheet material.

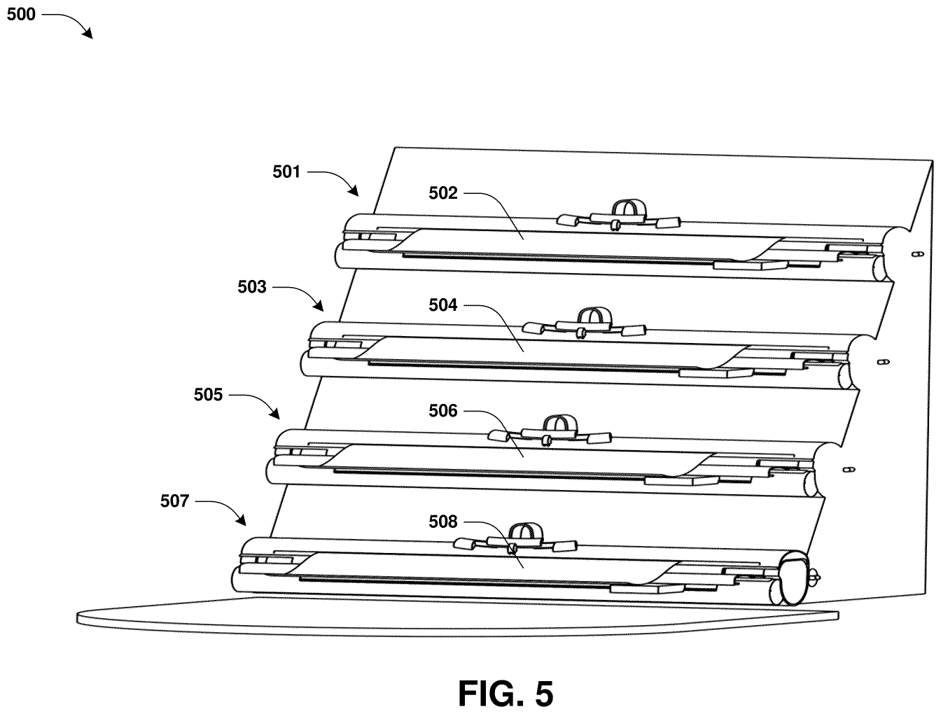

FIG. 5 illustrates a multifunctional wrapping apparatus 500 comprising a plurality of dispensing assemblies (e.g., a first dispensing assembly 501, a second dispensing assembly 503, a third dispensing assembly 505 and/or a fourth dispensing assembly 507) for cutting and wrapping different sheets, in accordance with some embodiments. In some examples, the plurality of dispensing assemblies may dispense one or more roll of sheet materials, in which the one or more roll of sheet materials may be utilized for one or more purposes. In some examples, the first dispensing assembly 501 may be used to dispense a portion of a first roll of sheet material 502. In some examples, the first roll of sheet material 502 may comprise a first sheet material comprising a first thickness and/or a first material associated with the first sheet material. In some examples, the second dispensing assembly 503 may be used to dispense a portion of a second roll of sheet material 504. In some examples, the second roll of sheet material 504 may comprise a second sheet material comprising a second thickness and/or a second material associated with the second sheet material. In some examples, the third dispensing assembly 505 may be used to dispense a portion of a third roll of sheet material 506. In some examples, the third roll of sheet material 506 may comprise a third sheet material comprising a third thickness and/or a third material associated with the third sheet material. In some examples, the fourth dispensing assembly 505 may be used to dispense a portion of a fourth roll of sheet material 506. In some examples, the fourth roll of sheet material 506 may comprise a fourth sheet material comprising a fourth thickness and/or a fourth material associated with the fourth sheet material.

In some examples, the wrapping apparatus of the present disclosure may aim to solve various roll cutting problems associated with a roll of sheet material and/or to provide one or more methods of using the roll of sheet material. In some examples, using the wrapping apparatus of the present disclosure may lead to preserving the nature and the environment and using less roll of sheet materials. In an example, upon using the wrapping apparatus of the present disclosure, a user may utilize one or more roll sheet materials instead of washing one or more dirty containers (e.g., one or more dirty dishes). For example, a user may utilize the wrapping apparatus of the present disclosure to avoid using drinkable water and/or tap water to wash dirty kitchenware. In some examples, using the wrapping apparatus of the present disclosure may be less costly and more nature-friendly due to not wasting any water source. In some examples, the wrapping apparatus of the present disclosure may be used in public places such as restaurants and reduce high costs of water filtration in large scale.

In some examples, a container (e.g., a dish) may be used to keep a portion of sheet material in a safe way. In an example, the roll sheet may be dispensed and/or cut in a short time period to save time. In some examples, one or more components and/or one or more devices may be added to the wrapping apparatus to allow at least one of water saving, energy saving and/or or money saving.

In some examples, the wrapping apparatus may vacuum the air between the sheet material and the container. In an example, the user may use the container easily for eating food and/drinks and avoid obligatory cost of water, electricity, gas, hygienic matters. For example, the time of washing and drying one or more dishes may be saved based on the user utilizing the container with the sheet material (e.g., cellophane).

In some examples, a wrapping apparatus includes a dispensing assembly and a vacuum sealing device. The dispensing assembly includes a sheet holder and a cutting device. The sheet holder is configured to hold a roll of sheet material and dispense a portion of the sheet material over a container. The cutting device is configured to separate a dispensed sheet including the portion of the sheet material from the roll of sheet material. The vacuum sealing device is configured to apply a suction force to a space between the dispensed sheet and the container to draw the dispensed sheet towards at least one of a surface of the container or an item in the container.

In some examples, the vacuum sealing device includes a pump, and a suction tube connecting the pump to a hole defined in the container. The pump applies the suction force to the space via the suction tube.

In some examples, the vacuum sealing device includes a container holder including an input device configured to trigger the pump to apply the suction force in response to a user input.

In some examples, the cutting device includes a cutter configured to cut the dispensed sheet, a cutter driving mechanism configured to drive the cutter, and an input device configured to trigger the cutter driving mechanism to move the cutter across an extent of the sheet material to separate the dispensed sheet from the roll of sheet material in response to a user input.

In some examples, the cutter driving mechanism includes a first pulley, a second pulley, a conveyor belt configured to connect the first pulley to the second pulley, a wagon coupled to the cutter via one or more bearings, a rail including a first guide rod and a second guide rod, a first sensor connected to a first side of the rail, a second sensor connected to a second side of the rail, and an electric motor configured to at least one of (i) rotate the first pulley (ii) rotate the conveyor belt (iii) move the wagon in the reciprocating path, or (iv) move the cutter in the reciprocating path. The rail provides a reciprocating path for the wagon and the cutter.

In some examples, the cutter driving mechanism includes a handle for manual operation of the cutter.

In some examples, the wrapping apparatus includes a casing configured to house the dispensing assembly and the cutting device.

In some examples, the casing includes a lid configured to house a power supply to supply power for operation of the wrapping apparatus, a chamber configured to house the cutting device, a lower covering configured to house the dispensing assembly and the chamber, and a sheet guider plate configured to hold at least a portion of the dispensed sheet of the roll of sheet material. The chamber includes a first trench allowing a cutter to move. The sheet guider plate includes a second trench overlying the first trench.

In some examples, the lid is connected to the lower covering via a spring-loaded hinge to provide a restoring force. The casing includes a lock configured to secure the lid to the lower covering. The lid includes a groove in contact with the dispensed sheet and configured to provide a path for the cutter to move and separate the dispensed sheet. The lid includes a handle configured to unlock the lid from the lower covering and rotate the lid around the spring-loaded hinge. The lid includes a thumb grip configured to hold the lid.

In some examples, the wrapping apparatus includes an ultraviolet (UV) sterilizer device including a UV container defining a UV chamber configured to store one or more utensils, and one or more UV lamps configured to emit UV light to disinfect the one or more utensils.

In some examples, a wavelength range of the UV light is between about 100 nanometers to about 400 nanometers, and/or between about 200 nanometers to about 209 nanometers.

In some examples, the sheet holder includes a roller configured to rotate the roll of sheet material, a first clip configured to hold at least a first portion of the roller, and a second clip configured to hold at least a second portion of the roller.

In some examples, the roller includes a first fixed rod with a first hole, a second fixed rod with a second hole, a rotatable rod coupled to the first bearing from one side and coupled to the second bearing from the other side, the first clip including a first spike, and the second clip including a second spike. The first fixed rod is coupled to a first bearing. The second fixed rod is coupled to a second bearing. The rotatable rod is configured to hold the roll of sheet material. The first spike enters the first hole. The second spike enters the second hole.

In some examples, the dispensing assembly includes a sheet pulling grip configured to pull the dispensed sheet. The sheet pulling grip includes a hand grip, a sheet straightener roller coupled to the hand grip and configured to flatten the dispensed sheet, a plurality of blades configured to hold the dispensed sheet, and a push button configured to move the plurality of blades.

In some examples, a wrapping apparatus includes a dispensing assembly and a heat blower sealing device. The dispensing assembly includes a sheet holder configured to hold a roll of sheet material and dispense a portion of the sheet material over a container, and a cutting device configured to separate a dispensed sheet including the portion of the sheet material from the roll of sheet material. The heat blower sealing device is configured to apply a compression force to the dispensed sheet to push the dispensed sheet towards at least one of a surface of the container or an item in the container.

In some examples, the heat blower sealing device includes a compressor, a heating element, a hot air nozzle, and a compression tube connecting the compressor to the hot air nozzle. The compressor applies the compression force to the space via the compression tube and the hot air nozzle.

In some examples, the heat blower sealing device includes an input device configured to trigger the compressor to apply the compression force in response to a user input.

Unless specified otherwise, “first,” “second,” and/or the like are not intended to imply a temporal aspect, a spatial aspect, an ordering, etc. Rather, such terms are merely used as identifiers, names, etc. for features, elements, items, etc. For example, a first object and a second object generally correspond to object A and object B or two different or two identical objects or the same object.

Moreover, “example” is used herein to mean serving as an instance, illustration, etc., and not necessarily as advantageous. As used herein, “or” is intended to mean an inclusive “or” rather than an exclusive “or”. In addition, “a” and “an” as used in this application are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, and/or variants thereof are used in either the detailed description or the claims, such terms are intended to be inclusive in a manner similar to the term “comprising”.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing at least some of the claims.

Various operations of embodiments and/or examples are provided herein. The order in which some or all of the operations are described herein should not be construed as to imply that these operations are necessarily order dependent. Alternative ordering will be appreciated by one skilled in the art having the benefit of this description. Further, it will be understood that not all operations are necessarily present in each embodiment and/or example provided herein. Also, it will be understood that not all operations are necessary in some embodiments and/or examples.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

What is claimed is:

1. A wrapping apparatus, comprising:
a dispensing assembly comprising:
a sheet holder configured to hold a roll of sheet material and dispense a portion of the sheet material over a container; and
a cutting device configured to separate a dispensed sheet comprising the portion of the sheet material from the roll of sheet material; and
a vacuum sealing device configured to apply a suction force to a space between the dispensed sheet and the container to draw the dispensed sheet towards at least one of a surface of the container or an item in the container.

2. The wrapping apparatus of claim 1, wherein:
the vacuum sealing device comprises:
a pump; and
a suction tube connecting the pump to a hole defined in the container; and
the pump applies the suction force to the space via the suction tube.

3. The wrapping apparatus of claim 2, wherein the vacuum sealing device comprises a container holder comprising an input device configured to trigger the pump to apply the suction force in response to a user input.

4. The wrapping apparatus of claim 1, wherein the cutting device comprises:
a cutter configured to cut the dispensed sheet;
a cutter driving mechanism configured to drive the cutter; and
an input device configured to trigger the cutter driving mechanism to move the cutter across an extent of the sheet material to separate the dispensed sheet from the roll of sheet material in response to a user input.

5. The wrapping apparatus of claim 4, wherein the cutter driving mechanism comprises:
a first pulley;
a second pulley;
a conveyor belt configured to connect the first pulley to the second pulley;
a wagon coupled to the cutter via one or more bearings;
a rail comprising a first guide rod and a second guide rod, wherein the rail provides a reciprocating path for the wagon and the cutter;
a first sensor connected to a first side of the rail;
a second sensor connected to a second side of the rail; and
an electric motor configured to at least one of (i) rotate the first pulley (ii) rotate the conveyor belt (iii) move the wagon in the reciprocating path, or (iv) move the cutter in the reciprocating path.

6. The wrapping apparatus of claim 5, wherein the cutter driving mechanism comprises a handle for manual operation of the cutter.

7. The wrapping apparatus of claim 1, comprising:
a casing configured to house the dispensing assembly and the cutting device.

8. The wrapping apparatus of claim 7, wherein the casing comprises:
a lid configured to house a power supply to supply power for operation of the wrapping apparatus;
a chamber configured to house the cutting device, wherein the chamber comprises a first trench allowing a cutter to move;
a lower covering configured to house the dispensing assembly and the chamber; and
a sheet guider plate configured to hold at least a portion of the dispensed sheet of the roll of sheet material, wherein the sheet guider plate comprises a second trench overlying the first trench.

9. The wrapping apparatus of claim 8, wherein:
the lid is connected to the lower covering via a spring-loaded hinge to provide a restoring force;
the casing comprises a lock configured to secure the lid to the lower covering;
the lid comprises a groove in contact with the dispensed sheet and configured to provide a path for the cutter to move and separate the dispensed sheet;
the lid comprises a handle configured to unlock the lid from the lower covering and rotate the lid around the spring-loaded hinge; and
the lid comprises a thumb grip configured to hold the lid.

10. The wrapping apparatus of claim 1, comprising:
an ultraviolet (UV) sterilizer device comprising:
a UV container defining a UV chamber configured to store one or more utensils; and
one or more UV lamps configured to emit UV light to disinfect the one or more utensils.

11. The wrapping apparatus of claim 10, wherein a wavelength range of the UV light is at least one of:
between about 100 nanometers to about 400 nanometers; or
between about 200 nanometers to about 209 nanometers.

12. The wrapping apparatus of claim 1, wherein the sheet holder comprises:
a roller configured to rotate the roll of sheet material;
a first clip configured to hold at least a first portion of the roller; and
a second clip configured to hold at least a second portion of the roller.

13. The wrapping apparatus of claim 12, wherein:
the roller comprises:
a first fixed rod with a first hole, wherein the first fixed rod is coupled to a first bearing;
a second fixed rod with a second hole, wherein the second fixed rod is coupled to a second bearing; and
a rotatable rod coupled to the first bearing from one side and coupled to the second bearing from the other side, wherein the rotatable rod is configured to hold the roll of sheet material;
the first clip comprises a first spike, wherein the first spike enters the first hole; and
the second clip comprises a second spike, wherein the second spike enters the second hole.

14. The wrapping apparatus of claim 1, wherein the dispensing assembly comprises:
a sheet pulling grip configured to pull the dispensed sheet, wherein the sheet pulling grip comprises:
a hand grip;
a sheet straightener roller coupled to the hand grip and configured to flatten the dispensed sheet;
a plurality of blades configured to hold the dispensed sheet; and
a push button configured to move the plurality of blades.

15. A wrapping apparatus, comprising:
a dispensing assembly comprising:
a sheet holder configured to hold a roll of sheet material and dispense a portion of the sheet material as a dispensed sheet over a container;
a cutting device configured to separate the dispensed sheet from the roll of sheet material, wherein the cutting device comprises:
a cutter configured to cut the dispensed sheet;
a cutter driving mechanism configured to drive the cutter; and
an input device configured to trigger the cutter driving mechanism to move the cutter across the dispensed sheet to separate the dispensed sheet from the roll of sheet material in response to a user input; and
a casing configured to house the dispensing assembly and the cutting device.

16. The wrapping apparatus of claim 15, wherein the sheet holder comprises at least one of:
a roller configured to rotate the roll of sheet material, wherein the roller comprises:
a first fixed rod with a first hole, wherein the first fixed rod is coupled to a first bearing;
a second fixed rod with a second hole, wherein the second fixed rod is coupled to a second bearing; and
a rotatable rod coupled to the first bearing from one side and coupled to the second bearing from the other side, wherein the rotatable rod is configured to hold the roll of sheet material;
a first clip comprises a first spike, wherein the first spike enters the first hole; and
a second clip comprises a second spike, wherein the second spike enters the second hole.

17. The wrapping apparatus of claim 16, wherein the dispensing assembly comprises:
a sheet pulling grip configured to pull the dispensed sheet, wherein the sheet pulling grip comprises:
a hand grip;
a sheet straightener roller coupled to the hand grip and configured to flatten the dispensed sheet;
a plurality of blades configured to hold the dispensed sheet; and
a push button configured to move the plurality of blades.

18. A wrapping apparatus, comprising:
a dispensing assembly comprising:
a sheet holder configured to hold a roll of sheet material and dispense a portion of the sheet material over a container; and
a cutting device configured to separate a dispensed sheet comprising the portion of the sheet material from the roll of sheet material; and
a heat blower sealing device configured to apply a compression force to the dispensed sheet to push the dispensed sheet towards at least one of a surface of the container or an item in the container.

19. The wrapping apparatus of claim 18, wherein:
the heat blower sealing device comprises:
a compressor;
a heating element;
a hot air nozzle; and
a compression tube connecting the compressor to the hot air nozzle; and
the compressor applies the compression force to the space via the compression tube and the hot air nozzle.

20. The wrapping apparatus of claim 19, wherein the heat blower sealing device comprises an input device configured to trigger the compressor to apply the compression force in response to a user input.
